(19) United States
(12) Patent Application Publication
(10) Pub. No.: US 2025/0082815 A1
(43) Pub. Date: Mar. 13, 2025

(54) IRRADIATION CROSSLINKING AND STERILIZATION OF SILICONE

(71) Applicant: PAUL HARTMANN AG, Heidenheim (DE)

(72) Inventor: Markus Johannes KETTEL, Heidenheim (DE)

(73) Assignee: PAUL HARTMANN AG, Heidenheim (DE)

(21) Appl. No.: 18/580,551

(22) PCT Filed: Jul. 20, 2022
(86) PCT No.: PCT/EP2022/070342

§ 371 Date: Jan. 18, 2024

(30) Foreign Application Priority Data
Jul. 20, 2021 (DE) 10 2021 118 759.1

(51) Int. Cl. A61L 15/26; A61L 15/58; B32B 5/18; B32B 27/06; B32B 27/28; B32B 27/40; C08G 77/20; C09J 183/04

(52) U.S. Cl. CPC A61L 15/26; A61L 15/58; B32B 5/18; B32B 27/065; B32B 27/283; B32B 27/40; C08G 77/20; C09J 183/04; B32B 2266/0278; B32B 2305/022; B32B 2305/72; B32B 2535/00; C09J 2481/00

(57) ABSTRACT

The present invention relates to a process for producing adhesive and sterile silicone, to the adhesive and sterile silicone as such and to a wound dressing comprising a layer of the adhesive and sterile silicone.

### FIELD OF THE INVENTION

The present invention relates to a process for producing an adhesive and sterile silicone, to the adhesive and sterile silicone, and to a wound dressing comprising a layer of the adhesive and sterile silicone.

### BACKGROUND OF THE INVENTION

The present invention relates to wound dressings that contain a silicone layer as an adhesive. Wound dressings must be sterilized during the production process and then packaged in a sterile manner in order to ensure a germ-free environment.

The sterilization of wound dressings can take place conventionally by steam sterilization, by heating in an autoclave or by radiation sterilization with ionizing radiation or by gas sterilization. Furthermore, the production is effected under aseptic conditions.

However, steam sterilization has the disadvantage that the water vapor used is not compatible with all materials in wound dressings, and in particular hydrogels cannot be sterilized by water vapor. For instance, water vapor uncontrollably increases the moisture level in hydrogels. Gas sterilization has the disadvantage that gas molecules such as ethylene oxide can become incorporated in the structure of the gels.

Radiation sterilization with ionizing radiation has the disadvantage that this method is not compatible with conventional silicone adhesives. Ionizing radiation causes conventional silicone adhesives to undergo physical changes and decomposition, as a result of which the silicone adhesive cures and loses its adhesive properties.

Therefore, the object of the present invention was to provide a process for producing sterile silicone which still has adhesive properties after radiation sterilization.

A further object was to provide an efficient and quick process for producing a layer or wound contact layer containing adhesive and sterile silicone or a wound dressing comprising such a layer or wound contact layer.

### SUMMARY OF THE INVENTION

This object was surprisingly achieved by a process for producing an adhesive and sterile silicone 2, comprising the irradiation of a silicone 1 with ionizing radiation with an energy dose of 5-60 kGy, wherein the silicone 1
(a) is obtainable by polycondensation of a composition comprising dimethyldichlorosilane and/or trimethylmonochlorosilane and units from the group consisting of
(i) monomethyltrichlorosilane, and/or
(ii) an element of the group consisting of maleic acid, fumaric acid and trans-3-hexenedioic acid, and mixtures thereof,
and/or
(b) is selected from the group consisting of vinyl methyl silicone, phenyl vinyl methyl silicone and fluoro vinyl methyl silicone.

A silicone 1 of this type can be sterilized by ionizing radiation without decomposing and/or losing its adhesive properties.

A further aspect of the invention relates to an adhesive and sterile silicone 2 obtainable or obtained by the abovementioned process.

A further aspect of the invention relates to a silicone layer containing the adhesive and sterile silicone 2 of the present invention.

A further aspect of the invention relates to a wound dressing comprising a layer of the adhesive and sterile silicone 2 of the present invention and a cover layer.

A further aspect of the invention relates to a process for producing the wound dressing according to the invention, comprising
(a) providing an arrangement comprising a layer comprising a silicone 1, and a cover layer and/or a hydrogel layer,
(b) optionally packaging the arrangement in packaging,
(c) irradiating the arrangement with ionizing radiation for curing and sterilization with an energy dose E of 5-60 kGy.

A further aspect of the invention relates to the use of an abovementioned silicone 1 in a wound contact layer, wherein the silicone 1 retains its adhesive properties during a sterilization with ionizing radiation for curing and sterilization with an energy dose E of 5-60 kGy.

### BRIEF DESCRIPTION OF THE DRAWINGS

FIG. 1 describes a wound dressing with a silicone layer (10) and a hydrogel layer (12).

In FIG. 2, a cover layer including closure tape (26), an optional intermediate layer (28), a hydrogel layer (22), a silicone layer (20) and a release liner (24) are arranged to form a wound dressing.

### DETAILED DESCRIPTION OF THE INVENTION

Preferred embodiments of the present invention are described in detail below. Each preferred embodiment may be realized on its own or in combination with further embodiments. Furthermore, the elucidations with respect to preferred embodiments relate to all aspects of the present invention, i.e. to the process for producing an adhesive and sterile silicone 2, to the adhesive and sterile silicone 2, to the silicone layer containing the adhesive and sterile silicone 2, to the wound dressing, the process for producing the wound dressing according to the invention and to the use of the silicone 1 in a wound contact layer.

One embodiment relates to a process, comprising the irradiation of a silicone 1 with ionizing radiation with an energy dose of 5-60 kGy, wherein the silicone 1
(a) is obtainable by polycondensation of a composition comprising dimethyldichlorosilane and/or trimethylmonochlorosilane and units from the group consisting of
   (i) monomethyltrichlorosilane, and/or
   (ii) an element of the group consisting of maleic acid, fumaric acid and trans-3-hexenedioic acid, and mixtures thereof,
and/or
(b) is selected from the group consisting of vinyl methyl silicone, phenyl vinyl methyl silicone and fluoro vinyl methyl silicone. As a result of the irradiation, the irradiated silicone 2 is formed from the non-irradiated silicone 1.

The ionizing radiation is preferably beta radiation or gamma radiation, particularly preferably beta radiation. Those skilled in the art can define a dose calibration model for the specific device, the geometry and the line speed, and for other well-known process parameters. Commercially available devices for generating electron beams are readily available. For example, a model CB300 electron beam generating device (available from Energy Sciences, Inc. (Wilmington, MA)). Generally, a carrier film (for example polyester terephthalate carrier film) is guided through a chamber. The chamber is usually purged with an inert gas, for example nitrogen, while the specimens are cured with the electron beam. Multiple passes through the electron beam sterilizer may be required. This includes at least two, at least three, at least four or more passes. Commercially available gamma irradiation devices include devices that are often used for gamma irradiation sterilization of products for medical applications. Cobalt-60 sources are suitable.

The silicone 1 is obtainable by polycondensation. The composition may be hydrolyzed in the presence of water in order to then polymerize.

The term “units from the group consisting of” refers to “monomer units from the group consisting of” or to an “element from the group consisting of”.

The terms vinyl methyl silicone (VMQ), phenyl vinyl methyl silicone (PVMQ) and fluoro vinyl methyl silicone (FVMQ) are familiar to those skilled in the art and are, inter alia, commercially available rubbers.

VMQ denotes silicones having methyl and vinyl groups and comprises for example a repeat unit of the following formula (1):

—[OSi(R¹R²)]—,

where R¹ is vinyl and R² is methyl.

VMQ is for example obtainable by polymerization of a composition comprising vinylmethyldichlorosilane. For this purpose, vinylmethyldichlorosilane can be hydrolyzed and the polycondensation of the silanol monomers results in VMQ.

PVMQ denotes silicones having phenyl, methyl and vinyl groups. PVMQ may comprise phenylmethylsiloxane, vinylmethylsiloxane and dimethylsiloxane as repeat units and comprise for example a repeat unit of the following formula (2):

—[OSi(R³R⁴)]—,

where R³ is vinyl or phenyl and R⁴ is methyl.

The term “polymerization” encompasses polycondensation, polyaddition and/or free-radical chain polymerization.

FVMQ denotes silicones having fluorine, methyl and vinyl groups. FVMQ may have trifluoropropylmethylsiloxane, vinylmethylsiloxane and dimethylsiloxane as repeat units. FVMQ may for example comprise a repeat unit of the following formula (3):

—[OSi(R⁵R⁶)]—,

where R⁵ is vinyl, fluorine or a mono-or polyfluorine-substituted C₁-C₃ alkane and R⁶ is methyl.

The term “silicones” encompasses silicone elastomers and silicone rubbers. Also encompassed are both RTV (“room-temperature-vulcanizing”) and HTV (“high-temperature-vulcanizing”) silicones.

In one embodiment, the silicone 1 has a vinyl content of 0.1-10 wt %. The vinyl content may preferably be determined by means of NMR spectroscopy.

For this purpose, triphenylmethane is used as internal standard. The signal of the methine proton of this standard appears at 5.45 ppm and the signal of the vinyl groups of the samples appears at 5.7-6.0 ppm. 20 mg is weighed into a small test tube on an analytical balance. The internal standard is weighed in in such amounts that the characteristic peaks of the two substances give approximately the same integrals. This mixture is then dissolved in 1 ml of deuterated chloroform and transferred into an NMR tube using a syringe. The tube is tightly sealed with a stopper and a parafilm. The samples are analyzed on a Bruker-AMX 400 spectrometer. The evaluation is performed by comparing the integrals of the vinyl group peaks with the integral of the methine proton peak of triphenylmethane.

Alternatively, the vinyl content may be determined by means of bromometric titration (see C. Harzdorf. Bestimmung von Si—H und Si-Vinyl in siliciumorganischen Substanzen [Determination of Si—H and Si-vinyl in organosilicon substances], Zeitschrift für Analytische Chemie [Journal of Analytical Chemistry], 276 (1975) 279-283). For this purpose, a sample is weighed into an iodine flask on an analytical balance and dissolved in 20 ml of CCl₄. The amount of sample is selected such that it comprises no more than 1 mmol of vinyl groups. 20 ml of a 2.5% mercury chloride solution in glacial acetic acid and 20.0 ml of 0.1 M bromine solution in glacial acetic acid are added. The sample is thoroughly mixed for 2 minutes. The flask is sealed (moisten ground glass joint with glacial acetic acid) and left to stand in the dark for 30 minutes. 2 g of Kl and 50 ml of H₂O are then added. The iodine formed is titrated with a 0.1 N sodium thiosulfate solution. A blind test is carried out under the same conditions.

In one embodiment, the silicone 1 has a vinyl content of 0.01-100 mmol/g, 0.1-50 mmol/g or 0.5-10 mmol/g.

In one embodiment, the silicone 2 has a degree of crosslinking of 50-100%. In a preferred embodiment, the silicone 2 has a degree of crosslinking of 60-95% or 65-80%.

In one embodiment, the silicone 1 has a degree of crosslinking of 0.5-60%. In a preferred embodiment, the silicone 1 has a degree of crosslinking of 1-50%, 5-40% or 10-30%.

The degree of crosslinking refers to wt % and may be determined according to the following method.

A test piece (mass A (mg) of the test piece) is cut out of a shock-absorbing film such that the mass is about 50 mg. The test piece is immersed in 30 cm³ of chloroform at 23° C., left to stand for 24 hours and then filtered through a 200-mesh wire gauze in order to collect the undissolved residue on the wire gauze. The undissolved residue is vacuum-dried and then the mass B (mg) of the undissolved residue is accurately weighed. The degree of crosslinking is calculated from the thus obtained value according to the following equation:

$$Degree of crosslinking ( mass \% ) = ( B / A ) 100$$

The silicone 1 may contain vinyl groups which crosslink during the irradiation with ionizing radiation. Furthermore, the crosslinking may be influenced by the addition of a peroxide, such as hydrogen peroxide. In one embodiment, the silicone 1 is irradiated with ionizing radiation in the presence of a peroxide compound, preferably hydrogen peroxide. 0.01-3 wt % of hydrogen peroxide is preferably used. In a preferred embodiment, peroxide crosslinkers may be dispensed with entirely. The term wt % refers here to the weight of hydrogen peroxide in relation to the weight of silicone 1 and hydrogen peroxide.

In one embodiment, the silicone 1 and/or the composition according to (a) further comprises an element from the group consisting of
(iii) divinyl ether, 1,4-butanediol divinyl ether, diethylene glycol divinyl ether and triethylene glycol divinyl ether, and mixtures thereof.

It is possible to influence the crosslinking and curing of the silicone 1 by incorporating the divinyl compounds from group (iii) as additional crosslinking bridges.

In one embodiment, the composition according to (a) comprises 10-98 amount % of dimethyldichlorosilane and/or trimethylmonochlorosilane. In a preferred embodiment, the composition according to (a) comprises 20-98 amount %, 30-95 amount % or 40-90 amount % of dimethyldichlorosilane and/or trimethylmonochlorosilane. The term amount % refers to the molar amount in relation to the total molar amount of the composition according to (a).

In a particularly preferred embodiment, the composition according to (a) comprises 10-95 amount %, 50-90 amount % or 70-80 amount % of dimethyldichlorosilane. In another particularly preferred embodiment, the composition according to (a) comprises 0.1-40 amount %, 5-30 amount % or 10-20 amount % of trimethylmonochlorosilane. In an alternative embodiment, the composition according to (a) contains 0 amount % of trimethylmonochlorosilane. In other words, it is alternatively preferred that the composition according to (a) does not contain any trimethylmonochlorosilane.

In one embodiment, the composition according to (a) comprises 0.1-10 amount % of monomethyltrichlorosilane, and the energy dose is 5-60 kGy, preferably 5-45 kGy, particularly preferably 10-20 kGy. In a preferred embodiment, the composition according to (a) does not comprise any monomethyltrichlorosilane. Monomethyltrichlorosilane leads to strong crosslinking and promotes the curing of the silicone 1 during the irradiation. It is therefore advantageous if the composition according to (a) comprises no or only small amounts of 0.1-10 amount % of monomethylchlorosilane.

In one embodiment, the composition according to (a) comprises 0.1-10 amount % of an element of the group consisting of maleic acid, fumaric acid and trans-3-hexenedioic acid, and mixtures thereof, and
the energy dose is 5-60 kGy, preferably 15-45 kGy, particularly preferably 20-40 kGy.

In one embodiment, the composition according to (a) comprises 0.1-10 amount % of an element of the group consisting of divinyl ether, 1,4-butanediol divinyl ether, diethylene glycol divinyl ether and triethylene glycol divinyl ether, and mixtures thereof, and the energy dose is 5-60 kGy, preferably 15-45 kGy, particularly preferably 20-40 kGy.

The stated combination of specific energy dose and specific constituents of the composition makes it possible to adjust the adhesive and adhesion properties of the silicone 2 in a controlled manner. In general, stronger adhesive and adhesion forces have the advantage that they ensure better and longer adhesion to the skin. At the same time, lower adhesive and adhesion forces enable easier (atraumatic) removal from the skin or their position can be corrected after application.

In addition to adjusting the energy dose, the irradiation rate can also have an influence on the properties of the silicone 2. In a preferred embodiment, the irradiation rate is 0.1-100.0 kGy/min, 1-90 kGy/min, or 10-60 kGy/min. In a particularly preferred embodiment, the irradiation rate is 0.1-100.0 kGy/s, 1-90 kGy/s, or 10-60 kGy/s. Such high irradiation rates can be achieved by β radiation, whereby a particularly fast and efficient process is made possible. This is advantageous in particular in the case of series production in high-throughput processes, where the step of crosslinking and/or sterilization can limit the speed of the overall process.

In one embodiment, the silicone 2 has an adhesion force of 0.4-4 N/cm² and is sterile in accordance with Eur. Ph. 9.00/2.06.01.00. In a preferred embodiment, the adhesion force is 0.05-50 N/cm², 0.8-3 N/cm² or 1-2 N/cm². The adhesion force may preferably be measured according to the following test protocol:

Use may be made of a tensile testing device or a similar device which is able to move the clamping jaw back and forth at a speed of 300 mm/minute with an accuracy of ±2%. The device must be able to measure forces of at least 20 newtons with an accuracy of ±2%. A plate of float glass must measure 25±0.5 mm×30±2.0 mm and be at least 3 mm thick. A metal pin is attached to the underside and in the middle of the plate. The size of the pin should be selected such that it can be clamped in the jaw of the tensile testing machine. The samples are strips of representative specimens of the material to be tested. The samples must be 25 mm wide and have a minimum length of 175 mm in the machine direction. The cut must be straight and clean. At least 5 samples per specimen should be tested. The test should be carried out at 23° C.±2° C. and 50±5% RH. The samples or test pieces must be conditioned for at least 4 hours before the test.

The two ends of the adhesive strip are grasped and a loop is formed, with the adhesive side facing out, by connecting the two ends to one another. The loop connection is clamped in the movable jaw of the tensile machine and 10 mm of the loop is left to hang down freely. The sides of the jaw must be protected from the adhesive. The glass plate is fixed with its metal pin in the fixed jaw, the 30-mm side being at right angles to the sample fold. The machine is started in order to bring the loop into contact with the glass plate at a speed of 300 mm/minute. When a region of approx. 25 mm×25 mm of the loop is in contact with the plate, the movement direction of the jaw is reversed in order to initiate the separation at 300 mm/minute. It is important that the movement direction is reversed as quickly as possible. The maximum value of the force required to completely detach the loop from the plate is measured. The adhesion is reported as the average value of the 5 measurements (without taking into account the initial peak) in newtons (per unit area).

If the abovementioned method proves to be unsuitable, the adhesion force may alternatively be measured according to the following test protocol.

Use is made of a pulling device which is able to separate a laminate at an angle of 180° at a speed of 300 mm/minute and with a tolerance of ±2%. A silicone layer with a minimum size of 450 mm×250 mm is provided and a strip of adhesive tape or a film with adhesive surface material is applied with light finger pressure to the silicone layer in the machine direction. A standard roller is rolled back and forth over the complex twice at a speed of approx. 10 mm/second. The test specimens are placed between two glass or metal plates and then left to stand at 23° C.±2° C. for 20 hours under a pressure of 6.86 kPa (70 g/cm²) in order to ensure good contact between the adhesive and the silicone layer. The test specimens are then removed and stored for at least 4 hours at 23° C.±2° C. and 50±5% RH. The test objects are placed onto the testing machine such that the front panel can be separated from the substrate at a 180° angle. The layer is pulled off at a pull-off angle of 180° at a speed of 300 mm/minute. The adhesion is reported as the average value of the 5 measurements in newtons (per unit area).

In an alternative embodiment, the silicone 2 has a maximum adhesion force Fmax of 0.3 to 4 N, preferably 0.5 to 3 N, more preferably 0.6 to 2.6 N, in particular 0.8 to 1.5 N, measured by a tack test. The tack test is described in the experimental part below.

One embodiment relates to adhesive and sterile silicone 2 obtainable by the process according to the invention.

One embodiment relates to a silicone layer containing the adhesive and sterile silicone 2. The layer thickness of the silicone layer is preferably 0.1-5 mm.

One embodiment relates to a wound dressing comprising a silicone layer according to the invention, wherein the silicone layer may be designed as a wound contact layer. In the context of the present invention, a wound dressing is understood to mean a product which is applied to a wound is provided in a ready-to-use form.

In one embodiment, the wound dressing of the present invention comprises a cover layer, wherein the cover layer preferably comprises polyurethane.

In one embodiment, the wound dressing of the present invention comprises a hydrogel layer as a wound contact layer containing a hydrogel matrix and/or a hydrogel foam, wherein the silicone layer is designed as an adhesive for the wound dressing. In one embodiment, the wound dressing comprises a hydrogel layer on a wound contact layer, wherein the hydrogel layer may be configured as a hydrogel matrix. In the context of the invention, the term hydrogel denotes a finely dispersed system composed of at least one solid phase and one liquid phase. This solid phase forms a spongy, three-dimensional network, the pores of which are filled by a liquid (lyogel) or a gas (xerogel). The two phases preferably penetrate one another completely. As a result of water absorption, the three-dimensional network can increase its volume through swelling without losing structural cohesion. A hydrogel may preferably be composed of a synthetic or natural material, preferably of a hydrophilic synthetic material. The hydrogel layer may be continuous or discontinuous. For example, it may be applied over the entire surface or have channels, holes or differently shaped openings. A discontinuous hydrogel layer can comprise a multiplicity of discrete hydrogel elements which can have the shape of circles, squares or other regular or irregular polygons. In a wound dressing according to the invention, a hydrogel layer according to the invention may be applied directly to the silicone layer. Equally, the hydrogel layer may also be applied with the aid of an adhesive for the purpose of better cohesion. It is possible to arrange further layers between the silicone layer and the hydrogel layer. In one embodiment, a layer comprising hydrophilic polyurethane foam is arranged between the silicone layer and the hydrogel layer. Furthermore, it is conceivable and advantageous if the wound dressing comprises a mesh-shaped hydrogel on the wound side. The hydrogel promotes a moist wound environment and thus accelerates the healing.

Wound dressings comprising a hydrogel matrix with a layer thickness of 0.1 to 5.0 mm have also proven to be particularly advantageous embodiments. In particular, a wound dressing according to the invention thus comprises a wound contact layer with a layer thickness of 0.1 to 5.0 mm, particularly of 0.5 to 5.0 mm and very particularly preferably of 0.5 to 3.0 mm. Wound dressings with such layer thicknesses exhibit, firstly, no wound adhesion and, secondly, the ability to take up a wound exudate released by a wound and to forward it to the absorbent layer. These layer thicknesses may be the same at every point of the wound contact layer or assume different values in different regions of the wound contact layer.

Further preferably, the hydrogel matrix may comprise channels, in particular conical channels, for the passage of liquids from the first to the second side. This can in particular provide an improved passage for wound exudate and minimize backflow in the direction of the wound. In this case, provision is particularly preferably made for the channels to have an elliptical or a circular cross section, i.e. for the channels to have a circular or elliptical opening both on the first side and on the second side of the hydrogel matrix, where the circular or elliptical opening on the first side and the second side are different in size. Provision may however also be made for the channels to have a triangular, rectangular, square, pentagonal, hexagonal or another polygonal cross section. Provision is very particularly preferably made here for the first side to have openings which is larger in comparison with the opening situated on the second side (conical design).

According to a further development of the invention, provision may also be made for the wound contact layer or the hydrogel matrix to have openings having a diameter of 0.5 to 5 mm. In particular, the wound contact layer or the hydrogel matrix has openings having a diameter of 1 to 3 mm. Very particularly preferably, the wound contact layer or the hydrogel matrix has, on the first side facing the wound, openings having a diameter of 1 to 3 mm, where the second side of the wound contact layer or of the hydrogel matrix is in direct contact with the polyurethane foam.

The wound dressings according to the invention are suitable for the treatment of wounds. The present invention therefore also encompasses wound dressings for the treatment of wounds. In particular, the present invention encompasses wound dressings for the treatment of chronic wounds such as decubitus ulcers, pressure ulcers, pressure sores, ulcus cruris venosum, venous ulcers, ulcus cruris arteriosum, arterial ulcers, wounds as a consequence of diabetic foot syndrome, neuropathic ulcers, but also wounds as a consequence of autoimmune diseases or of tumors (ulcerating tumors) or of radiation damage in the case of tumor therapy.

Wound dressings according to the invention are suitable for phase-appropriate wound therapy, in particular for the therapy of wounds in the granulation phase and/or the epithelialization phase.

In one embodiment, the hydrogel matrix comprises at least 3-95 wt %, in particular 5-90 wt %, of water.

The hydrogel matrix may comprise a polyurethane-polyurea copolymer. This polyurethane-polyurea copolymer can be formed in particular from a prepolymer having aliphatic diisocyanate groups and a polyethylene oxide-based polyamine. In particular, the polyurethane-polyurea copolymer can be formed from an isophorone diisocyanate-terminated prepolymer, a polyethylene oxide-based polyamine, and water. These hydrogel matrices are particularly well suited for storing water and delivering this water to a wound.

Further preferably, the water-containing hydrogel matrix may further comprise at least one polyhydric alcohol from the group of dihydric, trihydric, tetrahydric, pentahydric or hexahydric alcohols. In particular, the alcohol may be selected from the group of glycols, in particular ethylene glycol or propylene glycol, and sorbitol or glycerol or mixtures thereof. These alcohols are of excellent suitability as moisturizers and thus constitute a care component for the skin surrounding the wound. The hydrogel matrix or the hydrogel foam may comprise 1-50 wt %, preferably 10-30 wt %, of a polyhydric alcohol.

Furthermore, provision may be made for the water-containing hydrogel matrix to comprise in particular at least one salt. In particular, provision is made in this case for the hydrogel matrix to comprise an inorganic salt. Chlorides, iodides, sulfates, hydrogensulfates, carbonates, hydrogencarbonates, phosphates, dihydrogenphosphates or hydrogenphosphates of alkali and alkaline earth metals are particularly suitable in this connection. Very particularly preferably, the hydrogel matrix comprises sodium chloride, potassium chloride, magnesium chloride, calcium chloride or mixtures thereof. These salts are particularly good simulators of the electrolyte mixture in a wound serum released by a wound. Thus, a hydrogel matrix comprising these salts provides a wound with conditions that are particularly conducive to wound healing.

In this case, provision may be made for the hydrogel matrix to comprise 0 to 5 wt % of at least one salt. In particular, the hydrogel matrix comprises 0.1 to 3 wt % of a salt and very particularly preferably 0.5 to 1.5 wt % of a salt.

Overall, provision may be made according to the present invention for the water-containing hydrogel matrix to preferably comprise at least 20 wt % of water and at least 10 wt % of polyurethane-polyurea copolymer. A further preferred hydrogel matrix comprises at least 20 wt % of water and at least 15 wt % of polyurethane-polyurea copolymer. In this case, provision may further preferably be made for the hydrogel matrix to be formed from 6 to 60 wt % of a prepolymer having aliphatic diisocyanate groups, 4 to 40 wt % of polyethylene oxide-based polyamine, 0 to 50 wt % of a polyhydric alcohol, 0 to 5 wt % of at least one salt selected from the group consisting of sodium chloride, potassium chloride, magnesium chloride, calcium chloride or mixtures thereof, and at least 20 wt % of water.

Provision may further preferably be made for the hydrogel matrix to be formed from 6 to 30 wt % of a prepolymer having aliphatic diisocyanate groups, 4 to 20 wt % of polyethylene oxide-based diamine, 10 to 30 wt % of a polyhydric alcohol selected from the group consisting of propylene glycol and/or glycerol, 0.5-1.5 wt % of a salt selected from the group consisting of sodium chloride, potassium chloride, magnesium chloride, calcium chloride or mixtures thereof, and at least 30 wt % of water.

Provision may very particularly preferably be made for the hydrogel matrix to be formed from 6to 20 wt % of isophorone diisocyanate-terminated prepolymer, 4 to 15 wt % of polyethylene oxide-based diamine, 15 to 20 wt % of propylene glycol and/or glycerol, 0.5 to 1.5 wt % of a salt selected from the group of sodium chloride, potassium chloride, magnesium chloride, calcium chloride or mixtures thereof, and at least 40 wt % of water. This hydrogel matrix has a free absorption A2 (based on the water-containing hydrogel matrix) of at least 1 g/g and at most 5 g/g, provides a non-irritating, liquid-absorbing, cushioning, skin-like medium that affords protection against bacteria, and thus is particularly well suited as a wound contact layer.

In a preferred embodiment, the hydrogel matrix comprises 37 to 43 wt % of propylene glycol, a prepolymer having isophorone diisocyanate end groups (hereinafter referred to as “isocyanate”) and a polyethylene oxide-based diamine in a total amount of 12 to 16.5 wt %, 0 to 5 wt % of an inorganic chloride, and water as the remainder, where the ratio of the reactive groups of isocyanate to the amine groups of the diamine should be 1.25 to 1.35.

In order to obtain the desired properties of the water-containing hydrogel matrix, it is advantageous for the composition of the hydrogel matrix to comprise, firstly, a proportion of propylene glycol of 37 to 43 wt %. This is a significantly higher content of propylene glycol in comparison with the compositions usually described in the prior art. Secondly, the composition may comprise a prepolymer having isophorone diisocyanate end groups and a polyethylene oxide-based diamine in a total amount of 12 to 16.5 wt %, where a ratio (“NCO:NH2”) of the reactive groups of isocyanate (“NCO”) to the amine groups of the diamine (“NH2”) which is set to the range of 1.25 to 1.35 may be present. The hydrogel matrix further comprises 0 to 5 wt % of at least one inorganic chloride. It has been found that any departure from the composition disclosed in the invention provides a hydrogel having comparatively less favorable properties, for example a hydrogel having a tack which has been rated with a value of less than 3 or more than 4, a hydrogel having an undesirably high pH of more than 8 or a hydrogel which does not cure sufficiently.

Particularly advantageous hydrogels may be obtained if the composition comprises 37 to 43 wt % of propylene glycol, preferably 39 to 41 wt % of propylene glycol, and a prepolymer having isophorone diisocyanate end groups and a polyethylene oxide-based diamine in a total amount of 12 to 16.5 wt %, preferably 14 to 16 wt %, where a ratio of the reactive groups of isocyanate to the amine groups of the diamine which is set to the range of 1.27 to 1.33, ideally to 1.29 to 1.31, is present. Hydrogels of this type have excellent properties with regard to tack, pH and stability.

It has proven to be very favorable if the hydrogel matrix comprises 0.5 to 1.5 wt % of an inorganic chloride, the inorganic chloride preferably being sodium chloride. In particular, the hydrogel matrix comprises 0.9 wt % of sodium chloride.

In one embodiment, the hydrogel has a water content of at least 50 wt % based on the total weight of the hydrogel, where the hydrogel comprises at least one gel-forming polysaccharide, at least one acrylic acid derivative and an electrolyte mixture, where the electrolyte mixture comprises at least two different electrolytes.

According to one embodiment, the hydrogel preferably comprises at least one gel-forming polysaccharide selected from the group of cellulose derivatives or salts thereof, alginates or derivatives thereof, chitin or derivatives thereof or salts thereof or starch. The origin of the gel-forming polysaccharides is irrelevant in this case, that is to say that these gel-forming polysaccharides may be of plant or animal origin or may be produced synthetically, for example by microbiological processes. It is also possible to use polysaccharides that are of plant or animal origin and are modified by chemical synthesis.

Furthermore, provision is made for the hydrogel to have a dynamic viscosity of 5000 to 60 000 mPa s, in particular 5000 to 50 000 mPa s and very particularly 10 000 to 40 000 mPa s (measured using a CSR-10 Bohlin rheometer, cone spindle 4°/Ø 40 mm, gap distance 100 μm, oscillometric measurement, T=22-27° C.). Such a hydrogel can be distributed particularly well and uniformly for example with a spatula over and in a wound, has good cohesion even when absorbing wound exudate and does not flow out of a wound to be treated.

In a particularly preferred embodiment, the hydrogel comprises at least one gel-forming polysaccharide and an acrylic acid derivative, where the weight ratio of the polysaccharide(s) to the acrylic acid derivative in the hydrogel corresponds to the ratio of 20:1 to 1:1, in particular 15:1 to 1:1 and very particularly 10:1 to 1:1.

In a particularly preferred embodiment, the hydrogel has a conductivity of at least 4000 μS cm⁻¹, in particular at least 6000 μS cm⁻¹ and very particularly preferably 6000-20000 μS cm⁻¹. The conductivity can in particular be adjusted by the amount of electrolyte mixture. This adjustment is advantageous since the conductivity of a hydrogel depends on the components of the gel and their concentrations. For example, the conductivity depends on the type and the concentration of the gel-forming polymer used or on the type and the concentration of the polyol used. These components lower the conductivity of a hydrogel to varying degrees in comparison with a pure salt solution. The amount of electrolyte mixture must therefore be individually adapted to the other ingredients of the gel in order to set a specific conductivity.

In one embodiment, the wound dressing is designed as an island dressing and the layer of the adhesive and sterile silicone 2 is arranged between the cover layer and the hydrogel layer and protrudes beyond the hydrogel layer so that the layer of the adhesive and sterile silicone serves as an adhesive for the wound dressing. The process according to the invention also makes it possible to adjust the adhesive and adhesion proprieties of the silicone 2 such that the silicone 2 develops initial adhesion (what is known as tack). Such initial adhesion holds in place a wound dressing applied to the skin or wound surface until further (secondary) dressings (for example gauze bandage, adhesive film, etc.) have been applied, which are used to set the final attachment of the wound dressing. This can be advantageous in the case of inflamed wound edges or wounds that require frequent dressing changes. On account of the initial adhesion, the subsequent detachment of the wound dressing is simplified and less painful for the patient. Accordingly, the present invention also encompasses wound dressings having initial adhesion, where the initial adhesion is ensured by the silicone 2 in the wound dressing. In particular, it is possible to use low adhesion forces in the range of 0.05-7.5 N/cm² for initial adhesion, taking into account the size of the adhesive surface of a wound dressing.

In one embodiment, the hydrogel has a free absorption A₃ (measured in accordance with DIN EN 13726-1 (2002)) of at least 1 g/g and at most 10 g/g. The hydrogel layer preferably has a pH of 5-8 in order to improve the healing process. The hydrogel layer preferably comprises a buffer system. By using a buffer system, it is possible to keep the pH virtually constant. The hydrogel layer preferably has a layer thickness of 3.2-5.9 mm.

The hydrogel layer may also comprise antimicrobial substances and/or infection indicators.

The wound dressing may comprise further optional layers. For instance, the wound dressing may comprise a foam layer and/or an adhesive layer.

In one embodiment, the present invention relates to a process for producing the wound dressing according to the invention, comprising
(a) providing an arrangement comprising a layer comprising a silicone 1, and a cover layer and/or a hydrogel layer,
(b) optionally packaging the arrangement in packaging,
(c) irradiating the arrangement with ionizing radiation for curing and sterilization with an energy dose E of 5-60 kGy,
   wherein the silicone 1
(a) is obtainable by polycondensation of a composition comprising dimethyldichlorosilane and/or trimethylmonochlorosilane and units from the group consisting of
   (i) monomethyltrichlorosilane, and/or
   (ii) an element of the group consisting of maleic acid, fumaric acid and trans-3-hexenedioic acid, and mixtures thereof,
and/or
(b) is selected from the group consisting of vinyl methyl silicone, phenyl vinyl methyl silicone and fluoro vinyl methyl silicone.

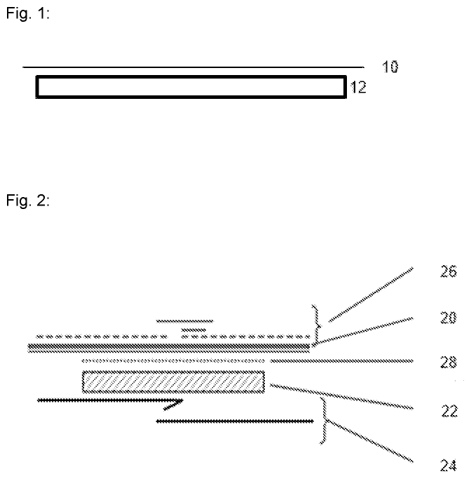

The term “arrangement” refers to a predetermined sequence of layers. An arrangement may contain the composition according to the invention. In addition, further layers may be present. These include a) a wound contact layer comprising or consisting of a hydrogel layer and/or a silicone layer according to the invention, b) an intermediate layer, c) a cover layer and d) an adhesive layer. The cover layer should always be positioned so as to be on the other side to the wound contact layer. Exemplary arrangements can be found in FIG. 1 and FIG. 2.

In one embodiment, the packaging and the irradiation take place on a support surface of a movement element, in particular of a conveyor belt.

In a preferred embodiment, the process according to the invention is a continuous process. The wound dressing is produced more efficiently on account of the sterilization by irradiation on a support surface of a movement element. For this purpose, the movement element can be stopped or run through a radiation source at a predetermined speed.

In particular, costs are reduced by such an “inline process” and the process is made more efficient as a whole. The assembly, packaging, crosslinking and sterilization can thus be carried out in one process step.

In this sense, the process according to the invention also encompasses an automated or partially automated process, which can comprise one or more features of the “inline method” mentioned.

The present invention further relates to the use of a silicone 1 in a wound contact layer, wherein the silicone 1 retains its adhesive properties during a sterilization with ionizing radiation for curing and sterilization with an energy dose E of 5-60 kGy, and wherein

the silicone 1
(a) is obtainable by polycondensation of a composition comprising dimethyldichlorosilane and/or trimethylmonochlorosilane and units from the group consisting of
   (i) monomethyltrichlorosilane, and/or
   (ii) an element of the group consisting of maleic acid, fumaric acid and trans-3-hexenedioic acid, and mixtures thereof,
and/or
(b) is selected from the group consisting of vinyl methyl silicone, phenyl vinyl methyl silicone and fluoro vinyl methyl silicone.

### Experimental Part

### Analysis: Tack Test to Determine the Maximum Adhesion Force Fₘₐₓ

The adhesion force of the silicone was tested on steel. A tensile testing machine which corresponded to standard DIN EN ISO 7500-01 was used. All measurements took place under standardized conditions of 23° C. and 50% relative humidity. The samples were attached to a horizontally movable support by means of double-sided adhesive tape. A metal weight having a weight force of 0.245 N and an underside made of glass was used to perform the measurement. The underside was cleaned with an ethanol-soaked pad before the start of the measurement. The weight was placed underside first on the silicone and the measurement was performed according to the parameters in Table 1:

TABLE 1

| Initial speed | Pull-off speed | Contact time |
|---|---|---|
| [mm / min] | [mm / min] | [s] |
| 100 | 400 | 2 |

After expiry of the contact time, the force required to pull off the weight at a 90° angle was measured using the tensile testing machine. The maximum adhesion force was determined three times in each case per sample/composition (see below) and then the average was formed and rounded to one decimal place.

### Examples: Production of Silicone 2

The silicone 2 was produced with different compositions, the production being performed according to the steps below:
Dichlorodimethylsilane and trichloromethylsilane and optionally chlorotrimethylsilane were mixed in a beaker.
Ether (1,4-butanediol divinyl ether) was optionally added to the silane mixture.
Distilled water or the aqueous acid solution (2 mg of maleic acid in 100 ml of water or 0.1 mg in 100 ml of water) was then added dropwise to the mixture, where an exothermic reaction with gas and bubble formation began.
The aqueous silicone phase was separated from the two phases formed after the exothermic reaction using a Pasteur pipette and transferred to a Petri dish.
After being left to dry, the silicone was applied in a thin layer to a PU film using a spatula and the corresponding samples were exposed to no irradiation or irradiation of different strengths.
Measurement of Fₘₐₓ of the different samples by way of the tack test described above

The different compositions, the irradiance and the maximum adhesion force are collated in Table 2 below.

TABLE 2

| | | | | Maleic | Fumaric | | | | | | |
|---|---|---|---|---|---|---|---|---|---|---|---|
| | | | | acid | acid | | | | | | |
| | | | | solution | solution | | | | | | |
| | | | | (2 g of | (0.1 g of | | | | | | |
| | | | | maleic | fumaric | | | | | | |
| | Dichloro- | Trichloro- | Pure | acid in | acid in | | Chloro- | Fmax | Fmax | Fmax | Fmax |
| | dimethyl- | methyl- | dist. | 100 ml | 100 ml | | trimethyl- | at | at | at | without |
| Designation/ | silane | silane | water | of water) | of water) | Ether | silane | 5 kGy | 15 kGy | 25 kGy | irradiation |
| Number | [ml] | [ml] | [ml] | [ml] | [ml] | [ml] | [ml] | [N] | [N] | [N] | [N] |
| 1 | 5 | 1 | 2 | | — | — | — | 0.1 | 0.1 | 0.3 | 0.2 |
| 2 | 7 | 1 | 2 | | — | — | — | 0.8 | 1.3 | 0.3 | 0.8 |
| 3 | 9 | 1 | 3 | | — | — | — | 2.2 | 1.3 | 1.9 | 0.0 |
| 4 | 5 | 1 | — | 2 | | — | — | 0.0 | 1.1 | 0.1 | 0.1 |
| 5 | 7 | 1 | — | 2 | | — | — | 0.1 | 0.6 | 0.2 | 0.1 |
| 6 | 7 | 1 | — | 3 | | — | — | 0.6 | 0.3 | 0.3 | 0.3 |
| 7 | 7 | 1 | — | 4 | | — | — | 1.8 | 2.5 | 0.7 | 2.2 |
| 8 | 9 | 1 | — | 2 | | — | — | 0.1 | 0.4 | 0.0 | 0.1 |
| 9 | 7 | 1 | — | 3 | | 1 | — | 1.2 | 0.8 | 0.5 | 0.1 |
| 10 | 7 | 1 | — | 3 | | 2 | — | 0.4 | 0.5 | 1.0 | 0.5 |
| 11 | 7 | 1 | — | 3 | | 3 | — | 2.6 | 1.3 | 0.8 | 0.2 |
| 12 | 7 | 1 | — | | 4 | — | — | 1.0 | 0.5 | 0.1 | 0.9 |
| 13 | 7 | 1 | — | | 3 | 3 | — | 1.2 | 3.5 | 2.3 | 0.0 |
| 14 | 9 | 1 | 3 | | — | — | 1 | 0.0 | 0.0 | 0.0 | 0.0 |
| 15 | 9 | 1 | 3 | | — | — | 2 | 0.0 | 0.0 | 0.0 | 1.5 |
| 16 | 9 | 1 | 3 | | — | — | 3 | 0.0 | 0.0 | 0.0 | 0.0 |

As can be seen from the table listed above, it is possible to produce a silicone with adhesive properties which is compatible with radiation needed for sterilization.

1.-20. (Cancelled)

21. A process for producing an adhesive and sterile silicone, comprising irradiating a silicone with ionizing radiation with an energy dose of 5-60 kGy, wherein the silicone
(a) is obtained by polycondensation of a composition comprising dimethyldichlorosilane and/or trimethylmonochlorosilane and units from the group consisting of
(i) monomethyltrichlorosilane, and/or
(ii) an element of the group consisting of maleic acid, fumaric acid and trans-3-hexenedioic acid, and mixtures thereof.

22. The process for producing an adhesive and sterile silicone according to claim 21, wherein the silicone and/or the composition according to (a) further comprises an element from the group consisting of
(iii) divinyl ether, 1,4-butanediol divinyl ether, diethylene glycol divinyl ether, triethylene glycol divinyl ether, and mixtures thereof.

23. The process for producing an adhesive and sterile silicone according to claim 21, wherein the silicone has a vinyl content of 0.1-10 wt %.

24. The process for producing an adhesive and sterile silicone according to claim 21, wherein the silicone has a degree of crosslinking of 50-100%.

25. The process for producing an adhesive and sterile silicone according to claim 21, wherein the composition comprises 10-98 amount % of dimethyldichlorosilane and/or trimethylmonochlorosilane.

26. The process for producing an adhesive and sterile silicone according to claim 21, wherein the composition comprises 0.1-10 amount % of monomethyltrichlorosilane, and the energy dose is 5-60 kGy.

27. The process for producing an adhesive and sterile silicone according to claim 21, wherein the composition comprises 0.1-10 amount % of an element of the group consisting of maleic acid, fumaric acid and trans-3-hexenedioic acid, and mixtures thereof, and the energy dose is 5-60 kGy.

28. The process for producing an adhesive and sterile silicone according to claim 21, wherein the composition comprises 0.1-10 amount % of an element of the group consisting of divinyl ether, 1,4-butanediol divinyl ether, diethylene glycol divinyl ether and triethylene glycol divinyl ether, and mixtures thereof, and the energy dose is 5-60 kGy and/or wherein the silicone has an adhesion force of 0.4-4 N/cm² and is sterile in accordance with Eur. Ph. 9.00/2.06.01.00.

29. The process for producing an adhesive and sterile silicone according to claim 21, wherein the silicone is further selected from the group consisting of vinyl methyl silicone, phenyl vinyl methyl silicone and fluoro vinyl methyl silicone.

30. An adhesive and sterile silicone obtainable by the process according to claim 21.

31. A silicone layer containing adhesive and sterile silicone according to claim 30.

32. A wound dressing comprising a silicone layer according to claim 31, wherein the silicone layer is designed as a wound contact layer.

33. The wound dressing according to claim 32 further comprising a cover layer, wherein the cover layer comprises polyurethane.

34. The wound dressing according to claim 32, also comprising a hydrogel layer on a wound contact layer, wherein the hydrogel layer is a hydrogel matrix.

35. The wound dressing as claimed in claim 34, wherein the hydrogel matrix comprises at least 3-95 wt % of water.

36. The wound dressing according to claim 34, wherein the hydrogel matrix comprises a polyurethane-polyurea copolymer.

37. The wound dressing according to claim 34, wherein the hydrogel matrix or the hydrogel foam comprise 1-50 wt % of a polyhydric alcohol.

38. The wound dressing according to claim 31, wherein the wound dressing is an island dressing and wherein the layer of the adhesive and sterile silicone is arranged between a cover layer and the hydrogel layer and protrudes beyond the hydrogel layer so that the layer of the adhesive and sterile silicone serves as an adhesive for the wound dressing.

39. A process for producing the wound dressing according to claim 32, comprising
(a) providing an arrangement comprising a layer comprising a silicone, and a cover layer and/or a hydrogel layer,
(b) optionally packaging the arrangement in packaging,
(c) irradiating the arrangement with ionizing radiation for curing and sterilization with an energy dose E of 5-60 kGy,
wherein the silicone
(a) is obtained by polycondensation of a composition comprising dimethyldichlorosilane and/or trimethylmonochlorosilane and units from the group consisting of
(i) monomethyltrichlorosilane, and/or
(ii) an element of the group consisting of maleic acid, fumaric acid, trans-3-hexenedioic acid, and mixtures thereof.

40. The process according to claim 39, wherein the packaging and the irradiation take place on a support surface of a movement element.

41. The process according to claim 39, wherein the silicone is selected from the group consisting of vinyl methyl silicone, phenyl vinyl methyl silicone, and fluoro vinyl methyl silicone.

42. The use of a silicone in a wound contact layer, wherein the silicone retains its adhesive properties during a sterilization with ionizing radiation for curing and sterilization with an energy dose E of 5-60 kGy, and wherein the silicone
(a) is obtained by polycondensation of a composition comprising dimethyldichlorosilane and/or trimethylmonochlorosilane and units from the group consisting of
(i) monomethyltrichlorosilane, and/or
(ii) an element of the group consisting of maleic acid, fumaric acid, trans-3-hexenedioic acid, and mixtures thereof.

43. The use as claimed in according to claim 41, wherein the silicone is further selected from the group consisting of vinyl methyl silicone, phenyl vinyl methyl silicone and fluoro vinyl methyl silicone.

44. The process for producing an adhesive and sterile silicone according to claim 21, wherein the energy dose is 5-45 kGy.

45. The process for producing an adhesive and sterile silicone according to claim 21, wherein the energy dose is 10-20 kGy.
